(19) United States
(12) Patent Application Publication
(10) Pub. No.: US 2023/0077125 A1
(43) Pub. Date: Mar. 9, 2023

(54) METHOD FOR DIAGNOSING AGE-RELATED MACULAR DEGENERATION AND DEFINING LOCATION OF CHOROIDAL NEOVASCULARIZATION

(71) Applicant: Taipei Veterans General Hospital, Taipei City (TW)

(72) Inventors: Shih-Hwa CHIOU, Taipei City (TW); Shih-Jen CHEN, Taipei City (TW); De-Kuang HWANG, Taipei City (TW); Kao-Jung CHANG, Taipei City (TW); Yi-Ping YANG, Taipei City (TW); Yueh CHIEN, Taipei City (TW)

(73) Assignee: Taipei Veterans General Hospital, Taipei City (TW)

(21) Appl. No.: 17/939,785

(22) Filed: Sep. 7, 2022

Related U.S. Application Data
(60) Provisional application No. 63/241,421, filed on Sep. 7, 2021.

(51) Int. Cl. G06T 7/00; G06T 7/11; A61B 5/00

(52) U.S. Cl. CPC G06T 7/0012; G06T 7/11; A61B 5/7275; G06T 2207/10101; G06T 2207/20081; G06T 2207/30041; G06T 2207/30101

(57) ABSTRACT

The present disclosure pertains to a method for diagnosing AMD comprising receiving OCTA image of a subject, pre-processing the OCTA image to obtain image data, inputting the image data to a trained deep learning (DL) network, generating using the trained DL network an output that characterizes the health of the subject with respect to AMD, and generating a diagnostic result based on the output.

### CROSS REFERENCE TO RELATED APPLICATIONS

This application claims priority to U.S. Provisional Application No. 63/241,421 filed Sep. 7, 2021, the entire contents of which are hereby incorporated by reference.

### FIELD OF THE INVENTION

The present invention is directed to a computer-implemented diagnosing method for classifying age-related macular degeneration (AMD), detection of neovascularization (NV) and keen surveillance on vessel leakage degeneration.

### BACKGROUND OF THE INVENTION

Age-related macular degeneration (AMD) is a prevalent (8.7%) disease that causes vision loss in developed countries, meanwhile the exudative AMD (exAMD) form requires imminent intervention, and is accounted for 10% to 15% of the AMD populations [1]. The visual threat of exAMD is majorly resulted by choroidal neovascularization (NV) and endothelium exudates. In current medication standards, exAMD was not curable but is primarily controlled by the expensive anti-vascular endothelial growth factor (anti-VEGF) biologics. The indication of anti-VEGF treatment to retinal NV was guided by non-vascular biomarkers, presence of intra/subretinal fluid [2] and subretinal hyper-reflective material (SHRM) [3] on optical coherence tomography (OCT) images were regarded as the signs of neovascular activities. However, the method is time-consuming and lacks of an objective standard when specialists were requested to monitor neovascular changes from surrogate biomarkers that consist no vascular information within [4, 5].

Deep learning (DL) uses convolutional neural networks as a feature extraction framework to recognize disease patterns from medical images. To date, using retinal fundus images and OCT scans, deep learning platforms were able to identify referable patients [6] and had achieved specialist comparable inspection standard in AMD classification [6-9]. Moreover, a parallel study of DL in color fundus pictures had shown visual impairment in the AMD patients were predictable [10]. The avant-garde breakthroughs of deep learning demonstrated in AMD studies had sparked clinical and research interest to explore questions that were not investigable by canonical approaches.

Optical coherence tomography angiography (OCTA) provides high-resolution images to visualize blood vessels down to the capillary level. exAMD is a disease context with primarily neovascularization conditions, and the advantage of applying OCTA to macular degeneration is the gain of projection resolved vascular plexus, whereby the distinct superficial capillary plexus (SCP), deep capillary plexus (DCP), choroid capillary (CC) vasculature structures and specific pathological NV lesions at designated retinal depth can be illustrated in detail. en face angiogram of plexuses is essential in OCTA analysis as NV membrane may encompass only a single plexus or may manifest differently in individual plexuses even when it is involved in multiple [12]. The major challenge for DL exists in the need to obtain a large quantity of annotated OCTA database and the difficulty of interaction with any single layer of the network, which can contribute to the view of deep networks as black-boxes, which hinders the explanation of their predictions in a manner easily understandable by humans.

Accordingly, it is still desirable to have an accurate and easily-conducting method for early AMD diagnosis through new technology or system.

### SUMMARY OF THE INVENTION

The present invention pertains to a computer-implemented diagnosing method for classifying age-related macular degeneration (AMD) by combining an optical coherence tomography angiography (OCTA) retinal image with deep learning (DL) procedure to explore how machine interprets vascular morphology.

In one aspect, the present invention provides a computer-implemented method for diagnosing AMD, the method comprising: receiving one or more optical coherence tomography angiography (OCTA) image of a subject; pre-processing the one or more OCTA image to obtain image data; inputting the image data to a trained deep learning (DL) network; generating, using the trained DL network, an output that characterizes the health of the subject with respect to AMD; and generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

In another aspect, the present invention provides a system comprising one or more computers and one or more storage devices storing instructions that when executed by the one or more computers cause the one or more computers to perform operations comprising: receiving one or more optical coherence tomography angiography (OCTA) image of a subject; pre-processing the one or more OCTA image to obtain image data; inputting the image data to a trained deep learning (DL) network; generating, using the trained DL network, an output that characterizes the health of the subject with respect to AMD; and generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

In a further aspect, the present invention provides one or more non-transitory computer-readable storage media encoded with instructions that when executed by one or more computers cause the one or more computers to perform operations comprising: receiving one or more optical coherence tomography angiography (OCTA) image of a subject; pre-processing the one or more OCTA image to obtain image data; inputting the image data to a trained deep learning (DL) network; generating, using the trained DL network, an output that characterizes the health of the subject with respect to AMD; and generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

In some embodiments, the pre-processing comprises segmenting the OCTA image to obtain at least one of an image of superficial capillary plexus, an image of deep capillary plexus, an image of outer retinal layer, and an image of choroid capillary layer.

In some embodiments, the output is generated based on image data of at least the image of deep capillary plexus.

In some embodiments, the output is generated based on image data of at least the image of deep capillary plexus and the image of outer retinal layer.

In some embodiments, a plurality of training OCTA images is used in training the DL network, each training OCTA images being pre-processed by segmenting training OCTA image to obtain at least one of an image of superficial capillary plexus, an image of deep capillary plexus, an image of outer retinal layer, and an image of choroid capillary layer. According to certain embodiments of the present invention, the DL network is trained with image data of the image of superficial capillary plexus, the image of deep capillary plexus, the image of outer retinal layer, and the image of choroid capillary layer.

In some embodiments, the classification of AMD classifies the subject as having no AMD, wet AMD or dry AMD.

In some embodiments, a customized convolution neural network (CNN) architecture is constructed to analyze multiple layer images and extract the different biomarkers as a novel method to resolve the early diagnosis of AMD and vascular leakage detection; the method comprising generating a deep learning (DL) classifier that classifies ophthalmic medical data, including image data, into one of a plurality of classifications, wherein the deep learning (DL) classifier is generated by training a convolutional neural network (CNN) using a customized dense block-based neuron network on the angiographic and en-face inputs including deep capillary plexus (DCP) and other specific layers; obtaining an ophthalmic image of an individual; evaluating the ophthalmic image using the deep learning (DL) classifier to generate a determination of the classification of age-related macular degeneration (AMD), detecting the presence of neovascular (NV) and neovascular (NV) activity, ophthalmic disorder, or condition, the determination having a sensitivity greater than 90% and a specificity greater than 90%. The other specific layers may include superficial capillary plexus (SCP), outer retinal layer, and the choroid capillary layer.

In some embodiments, to fully explore the diagnostic power of optical coherence tomography angiography (OCTA), in association with deep learning to further develop a new methodology for diagnosis and characterization of age-related macular degeneration (AMD), and detection on vessel activities like neovascularization (NV) wherein evaluating the ophthalmic image comprises uploading the ophthalmic image to a cloud-based network for remote analysis of the ophthalmic image using the deep learning (DL) classifier.

The features and advantages of the present invention will be apparent to those skilled in the art. While numerous changes may be made by those skilled in the art, such changes are within the scope of this invention.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 provides the demographic results of our AMD featured OCTA databank. (A) The protocol of AMD-OCTA databank collection. OCTA images with non-AMD etiologies were excluded, additional color fundus and dye-based angiography were enrolled to assist AMD confirmation. Based upon the presence of NV and retinal fluid, a total 1714 AMD-OCTA pictures were classified into distinct AMD subtypes. (B) The LogMAR visual loss of each AMD patient subgroups were calculated, and the NV positive wet-AMD (exudative and quiescent AMD) had more significant visual loss than the controlled groups (p=1.3E-04***). Furthermore, the vision loss in exudative subgroup is even significant than the quiescent subgroup (p=0.041*). (C) The age discrepancy is non-significant (p=0.35) between the quiescent and exudative AMD subgroup.

FIG. 2 shows the deploying cloud-based DL to monitor neovascular changes in retinal degeneration. (A) An exudative AMD case with subretinal fluid (indicated by blue arrow) was pictured in cross OCT, the degree of fluid accumulation can be further reflected by the thickness of the retina. When the same lesion was depicted on OCTA, a prominent fluid related hypo-density signal (circled by blue dashed line) was seen at the outer retina en-face, meanwhile the sprouting NV meshwork (circled in dashed line) was also evident on angiogram. (B) A same set of image modality was repeated to record treatment response after the patient received intra-vitreous anti-VEGF injections. Collectively, the subretinal fluid was absorbed, retina thickness returns to normal range. While the hypo-dense region regressed from en-face, the NV network appears indistinguishable on the angiogram. (C) After another 5 months, the exudative NV recurred, which give rise to subretinal fluid accumulation, retinal thickening and hypo-density on OCTA en-face. (D) To automize and standardize the multi-modal AMD evaluation protocol, we proposed a cloud-based DL to process patient volumetric OCTA pictures. The presence of NV may be simultaneously evaluated for its exudate activity.

FIG. 3 shows the inspect vascular and structural changes in early and late stage of retinal degeneration. (A) In the early phase of retina exudation, the color fundus image depicted a pool of peri-macular fluid, which was later confirmed by dye-based fluorescein angiography. In the cross OCT and OCTA, the intraretinal fluid manifested with a canonical hypo density feature. (B) Within a three-year interval, plenty exudative recurrence had reshaped the retina. Geographic atrophy of the pigment epithelium as well as severe macular scare was noted respectively on the color fundus and cross OCT. Meanwhile the vascular signal on OCTA becomes fuzzy and distorted from regular appearance. (C) In aim to dissect the influence of anatomy variance to the DL performance, we designate the AMD pictures taken within one year of first diagnosis, an independent experiment was done with the “early AMD” subset (n=275) apart from the bulk data (n=1029). (D) From the longitudinal history of the AMD patient care, revisit count (x) was plotted against the prevalence of each AMD subtype (y), wherein we observe higher follow-up adherence was associated with less exudative AMD (y=−0.0579×ln(x)+0.5077) and higher quiescent AMD (y=0.0819×ln(x)+0.2947). (E) Avoiding training-validation data contamination, images from a single patient were bundled as a unit before fed into K-fold validation experiments.

FIG. 4 shows the model structure and Input combinations; the AMDenseNet was established by tuning a customized dense block-based neuron network on the angiographic and en-face inputs cropped from each anatomy plexus imaged on the OCTA. After vascular features were extracted by the neuron units, the final classification results were made by global average pooling and softmax layer. The axiom attribution module (heat map) was drawn by a concatenated function to the AMDenseNet.

FIG. 5 shows the investigation of anatomy dependence underlying the model decision of AMD classification. (A) The deploy of deep learning in the screening and follow-up of both the undiagnosed and previous-diagnosed AMD patients. (B) and (C) The ROC curves of experiment models classifying AMD features ((B): NV presence and (C): NV exudation) were plotted. (D) The plexus specific contribution to model performance was investigated by layer removal. (E) and (F) The split layer model ROC curves ((E): NV presence and (F): NV exudation). (G) The area under ROC curve was further calculated, whereby the model anatomy dependence was defined by the drop of model performance upon layer removal. (H) A parallel kappa score matrix was calculated to address anatomy contribution to model decision in characterizing NV activity. Numerical code: 1=Angio-SCP; 2=Angio-DCP; 3=Angio-Outer Retina; 4=Angio-Choroid Capillary.

FIG. 6 shows the investigation of the en-face dependence underlying the model decision of AMD classification. Model performance resulting from structural en-face input combinations were compared to the angiogram input results in FIG. 5. (A) The general model ROC curve detection for (B) NV presence and (C) NV exudation. Whereas the split layer model ROC curves detecting (C) NV presence and (D) NV exudation were also plotted. (E) Area under ROC curve was further calculated, whereby the model anatomy dependence was defined by the drop of model performance upon layer removal. The model decisions were investigated by inter-layer consistency test, a kappa score matrix was calculated to address anatomy contribution to model detecting the (F) presence and the (G) activity of the NV. Numerical code: 5=enface-SCP; 2=enface-DCP; 3=enface-Outer Retina; 4=enface-Choroid Capillary.

FIG. 7 shows that applying DL to grade retinal NV risk and associated vision loss by angiographic inputs of AMID. (A) The DL graded AMD risk on the OCTA pictures corresponds with manual annotated NV size and maturity. (B) The scale of DL graded AMD risk positively correlates with the clinic measured vision loss. (C) Investigation of the necessity of each vascular plexus in the function of DL predict visual loss, DL experiments were re-tested by removing one vascular layer at a time from the model input. The vascular significance underlying the model decision was evaluated by back propagated function loss. (D) The hex bin plot depicts the distribute relation between vision loss and DL graded risk. The remove of deep vascular plexus from DL input results in an under-estimated risk for those with greater vision loss.

FIG. 8 shows that applying DL to grade retinal NV risk and associated vision loss by en-face inputs of AMD. As a collateral investigation to FIG. 7, we examined the necessity of each en-face plexus in the function of DL predict visual loss. DL experiments were tested by removing one structural plexus layer at a time from the model input. The plexus significance underlying each model decision was evaluated by back propagated function loss.

FIG. 9 shows that applying DL to assess real world treated or reactivated exudative AMDs. (A) To evaluate the reliability of OCTA based DL algorithm in real world NV evaluations, we compare model performance to medical raters ranging from variable profession backgrounds. (B) In 66 paired test images of inactive-to-active transitioned NV showed visual loss (p=0.0009). (C)

In the reactivated NV pairs, machine predicted active NV probability increased from 0.67 to 0.96. (D) The AI matches with medical workers in paired NV reactivation OCTA test. (E) In 93 paired test images of active-to-inactive transitioned NV showed restored vision (p=0.04). (F) In the treatment remission NV pairs, machine predicted active NV probability decreased from 0.98 to 0.51. (G) The AI matches with medical workers in paired NV treatment remission OCTA test.

FIG. 10 demonstrates DL robustness in assessing recurrent AMD with longitudinal OCTA records. (A) The schematic drawing of an exAMD case receiving regular care, during which the OCTA pictures and other standard examinations alongside IVI treatment events were recorded. For each visit date, the patient NV exudative status was specified, whereby the red pinhead represents for exudative AMD; blue ones for the quiescent AMD; and the green triangle marks down the time of anti-VEGF intervention. From the excerpted record interval, three clinical sequences (i, ii and iii) were referenced to elaborate the strength of combining DL with OCTA in AMD care. (B) To elucidate whether the vascular information (angiogram) or the structural features (retinal thickness and retinal exudates) could better depict the onset of NV exudation, we compare each module sensitivity to NV changes at distinct phases of disease progression. And DL-OCTA denotes NV exudative changes before signs of retina thickening or fluid exudates. (C) During the six months interval of clinical scenario (i), the exudative AMD was treated and again recurred. While the DL-OCTA captures the NV status change with corresponding active NV probability, the retina thickness remains at an invariable range: 233-244(um). (D) Disease impressions were made by DL-OCTA and specialist-thickness map/OCT cross sections. A diagnose mismatch between the DL and specialists can be noticed in the first and the fourth image sets, wherein the thickness map remains silent while the angiographic changes were depicted by DL, and was attributed correct exAMD diagnosis. (E) Similarly, in clinical scenario (iii) the remission and recur of the exAMD was recorded by OCTA and retinal thickness. (F) Diagnosis mismatch remains at the second and fourth time point, which the specialists infer the thickened (red coded) area possess residual NV exudates. For retinal thickness map, each color encodes the thickness percentile rank among general population, red: >99%; yellow: >95%; green: >5%; light blue: >1%; dark blue: <1%.

FIG. 11 shows the axiom attribution in DL processing identifies feeder vessels supplying for NV exudates. (A) The schematic drawing of the cloud-based DL locates layer specific vascular leakage. Aa demonstrated in the provided case (FIG. 11, (B)), the XAI-OCTA identifies feeder vessel upstream to NV exudates. (B) An exudative case progression was denoted in a panel of OCT, FA and OCTA images. By combining OCT and FA, we locate the retinal exudate at the depth between IPL and INL (indicated by a blue arrow) while the leakage vessel was at the supra-temporal site (marked by blue dashed line) to the hyper fluorescent pooling of RPED. However, the exact depth and branch of the involved vascular lesion was not clarified. To this end, axiom attribution in XAI-OCTA had converged and marked somewhat as-per the algorithm's view, the important features for classifying exudative NV progression. And such DL attributed vascular region in SCP layer perfectly align to the “feeder vessels” in a clinical sense. (C) To further validate the axiom attribution by DL was not a consequence of random assignment, and such decision was based upon anatomy inferences, we recorded the XAI-OCTA attribution pattern with incomplete vascular inputs. (D) A clinical sequence of exAMD-qAMD-exAMD was provided and confirmed by the cross OCT and retinal thickness changes. (E) With full vascular layer inputs, the XAI-OCTA locates and attributes exudative status of the neovascular net at the depth of outer retina. (F) By removing DCP from the model input, the XAI-OCTA went blind to the vascular network, and could neither attribute image features that represents the exudation of NV.

### DETAILED DESCRIPTION OF THE INVENTION

The above summary of the present invention will be further described with reference to the embodiments of the following examples. However, it should not be understood that the content of the present invention is only limited to the following embodiments, and all the inventions based on the above-mentioned contents of the present invention belong to the scope of the present invention.

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

Age-related macular degeneration (AMD) is one of the leading causes of global blindness. Early detection of neovascularization (NV) and keen surveillance on vessel leakage are the paramount to control the disease thus bring the optimized visual outcome. Optical coherence tomography angiography (OCTA) is a state-of-the-art technique which provides holistic three-dimension (3D) resolution to retinal vasculature structure without intravenous contrast injection. In recent, OCTA take part in AMD workup as a swift, non-invasive module that bypass cumbersome examine protocol and deadly allergy events seen in fundal fluorescein angiography (FAG) or indocyanine green imaging (ICG). Herein, we set to investigate the clinical value of OCTA could have grossed with the help of artificial intelligence (AI).

In one aspect, the present invention provides a computer-implemented method for diagnosing AMD, the method comprising:

receiving one or more optical coherence tomography angiography (OCTA) image of a subject;

pre-processing the one or more OCTA image to obtain image data;

inputting the image data to a trained deep learning (DL) network;

generating, using the trained DL network, an output that characterizes the health of the subject with respect to AMD; and

generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

In another aspect, the present invention provides a system comprising one or more computers and one or more storage devices storing instructions that when executed by the one or more computers cause the one or more computers to perform operations comprising:

receiving one or more optical coherence tomography angiography (OCTA) image of a subject;

pre-processing the one or more OCTA image to obtain image data;

inputting the image data to a trained deep learning (DL) network;

generating, using the trained DL network, an output that characterizes the health of the subject with respect to AMD; and

generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

As used herein, the term “neovascularization activity” or “NV activity” refers to an activity of choroidal neovascularization (CNV), which involves the growth of new blood vessels that originate from the choroid through a break in the Bruch membrane into the sub-retinal pigment epithelium (sub-RPE) or subretinal space. Said NV activity includes but is not limited to NV formation, and an NV status change or an NV exudative change.

“Assessing the risk of a subject developing a disease or condition” refers to the determination of the chance or the likelihood that the subject will develop the disease or condition. This may be expressed as a numerical probability in some embodiments. The assessment of risk may be by virtue of the extent of NV determined by methods of the invention.

As used herein, the term “wet AMD” may refer to NV positive wet-AMD, which includes exudative and quiescent AMD.

An OCTA image may be pre-processed by applying any of a variety of conventional image processing techniques to the image to improve the quality of the output generated by the machine learning model. As an example, a computer may be used to crop, scale, deskew or re-center the image. As another example, a computer may be used to remove distortion from the image, e.g., to remove blurring or to re-focus the image, using conventional image processing techniques.

Validation of the machine-learning diagnosis allows artificial neural network (ANN) to support the diagnosis by a physician or to perform diagnose, allows the physician to perform treatment based on the diagnosis, or allows the ANN to support the treatment by the physician or to perform the treatment.

A method for validating machine-learning may include creating an input that maximizes an ANN output (Activation maximization) method. For the ANN that deals with classification problems, the output is a classification probability for each category. Here, estimation of the reasons for determination may be performed by finding an input in which classification probability of a certain category is quite high, and specifying a “representative example” of the corresponding category by the ANN.

Alternatively, a method of Sensitivity Analysis for analyzing the sensitivity for the input may be used. That is, when the input feature amount has a large influence on the output, the input feature can be regarded as an important feature quantity, and the amount of change indicating which of the inputs the ANN is sensitive is examined. The amount of change can be determined by a gradient. Since the ANN learns by the gradient, ANN is well suited to an already available optimization mechanism.

The system may include a health analysis subsystem that receives the output and generates the diagnostic result. Generally, the health analysis subsystem generates a diagnostic result that characterizes the output in a way that can be presented to a user of the system. The health analysis subsystem can then provide the diagnostic result for presentation to the user in a user interface, e.g., on a computer of a medical professional, store the diagnostic result for future use, or provide the diagnostic result for use for some other immediate purpose.

In some embodiments, the diagnostic result also includes data derived from an intermediate output of the DL network or DL model that explains the portions of the OCTA image or images that the machine learning model focused on when generating the output. In particular, in some embodiments, the DL model includes an attention mechanism that assigns respective attention weights to each of multiple regions of an input OCTA image and then attends to features extracted from those regions in accordance with the attention weights. In these embodiments, the system can generate data that identifies the attention weights and include the generated data as part of the diagnostic result. For example, the generated data can be an attention map of the OCTA image that reflects the attention weights assigned to the regions of the image. For example, the attention map can be overlaid over the OCTA image to identify the areas of the subject's fundus that the DL model focused on when generating the model output.

The DL network may be a deep convolutional neural network and includes a set of convolutional neural network layers, followed by a set of fully connected layers, and an output layer. It will be understood that, in practice, a deep convolutional neural network may include other types of neural network layers, e.g., pooling layers, normalization layers, and so on, and may be arranged in various configurations, e.g., as multiple modules, multiple subnetworks, and so on.

In some embodiments, the DL network comprises one or more dense block layer comprising a depth-wise convolution sublayer and a point-wise convolution sublayer. In some embodiments, the DL network further comprises one or more convolution layer, one or more batch normalization layer, one or more rectified linear unit layer, one or more pooling layer, one or more global average pooling and softmax layer.

In some embodiments, a plurality of training OCTA images is used in training the DL network. Before use in training, each training OCTA images is subjected to an anatomy-based segmentation, which segments a training OCTA image into one of the four types: superficial capillary plexus, deep capillary plexus, outer retinal layer, and choroid capillary layer. According to certain embodiments of the present invention, the DL network is trained with image data of all the four types of images.

In some embodiments, the output is a set of scores, with each score being generated by a corresponding node in the output layer. As will be described in more detail below, in some cases, the set of scores are specific to particular medical condition. In some other cases, the each score in the set of scores is a prediction of the risk of a respective health event occurring in the future. In yet other cases, the scores in the set of scores characterize the overall health of the subject.

Generally, the set of scores are specific to a particular medical condition that the system has been configured to analyze. In some embodiments, the medical condition is AMD.

In some embodiments, the set of scores includes a single score that represents a likelihood that the patient has the medical condition. For example, the single score may represent a likelihood that the subject has AMD.

In some other embodiments, the set of scores includes a respective score for each of multiple possible levels or types of AMD, with each condition score representing a likelihood that the corresponding level is current level of AMD for the subject.

For example, the set of scores may include a score for no AMD, early-stage AMD, intermediate AMD, advanced AMD, and, optionally, an indeterminate or unspecified stage.

As another example, the set of scores may include a score for no AMD, wet AMD, and dry AMD.

The system may generate diagnostic result from the scores. For example, the system can generate diagnostic result that identifies the likelihood that the subject has AMD or identifies one or more AMD levels or types that have the highest scores.

The set scores may include a respective score for each of multiple possible levels of AMD, with each score representing a likelihood that the corresponding level will be the level of AMD for the subject at a predetermined future time, e.g., in 6 months, in 1 year, or in 5 years. For example, the set of scores may include a score for no AMD, early-stage AMD, intermediate-stage AMD, and advanced-stage AMD, and, optionally, with the score for each stage representing the likelihood that the corresponding stage will be the stage of AMD for the subject at the future time.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

Computers suitable for the execution of a computer program can be based on general or special purpose microprocessors or both, or any other kind of central processing unit. Generally, a central processing unit will receive instructions and data from a read only memory or a random-access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

The present invention provides in a further aspect one or more non-transitory computer-readable storage media encoded with instructions that when executed by one or more computers cause the one or more computers to perform operations comprising:

receiving one or more optical coherence tomography angiography (OCTA) image of a subject;

pre-processing the one or more OCTA image to obtain image data;

inputting the image data to a trained deep learning (DL) network;

generating, using the trained DL network, an output that characterizes the health of the subject with respect to AMD; and

generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

According to one Example of the present invention, an OCTA databank was established between year 2017 to 2020, all OCTA images were acquired by OptoVue RTVue-XR Avanti. 311 series of OCTA image studies from 133 patients were included for further analysis, of note poor quality images were not precluded from the dataset. In this collection: 52 normal controls without retinal abnormality, 24 drusen, 131 active neovascular age-related macular degeneration (nAMD), and 103 inactive nAMD patients were diagnosed by FAG/ICGA, all diagnoses were agreed by 2 retina specialists and serve as ground truth. The customized AMDenseNet convolution neuronal network (CNN) used in this invention was adopted from DenseNet-121 architecture and established on the Ubuntu 16.04 LTS operation system with the GeForce RTX 2080 Ti graphic processing unit (GPU) card. 10-fold cross validation was applied to overcome data scarcity. Keras 2.2.4 and TensorFlow-GPU 1.6.0 software were used for training and validation.

The AI model had sufficient comprehend to learn OCTA angiographic data and use it to discriminate AMD subtype (Normal vs. Wet vs. Dry). Among the tested combinations, the algorithm peaked its accuracy=0.910, precision=0.909, recall=0.91, F1 score=0.908 in work with input angio-outer retina+choroid capillary (aOR+aCC). Secondly, we further elaborate vascular morphology trained AI can perform wet-NV activity prediction as effective as the structural en-face trained counterpart (F1 score=91.7). Lastly, in two long term follow-up cases, explainable artificial intelligence (XAI) marks off subclinical microvascular lesions before the loci grew into noticeable NVs. Moreover, the time point of XAI NV prediction in a case is 1 month ahead of FAG/ICG examine; 6 months before NV visualized on OCTA, while in another case the XAI prediction came a month earlier than visual decline; and 8 months earlier than NV formation.

It can be concluded that early detect and precise typing of AMD vasculopathy is the breakthrough of this OCTA-AI study. The angiography images in our hands had demonstrated equivalent power as en-face images in discriminating AMD activity and typing, and this logistic can be learned by artificial intelligence. We hereby propose the context that OCTA in combine with AI could partly play the role of FAG/ICG in characterizing AMD-NV activity. In addition to the clinical value, this OCTA-AI study had taken the OCT-AI study a step forward, thus this study serves as a keystone to foster future AI work in novel optic images and diagnose modules.

In the present invention, AI-based diagnosis has been independently achieved for age-related macular degeneration (AMD) [21] and diabetic macular edema (DME) [22,30] by utilizing OCT images with accuracies was generally higher than 90%. In a subsequent DME study, we further demonstrated that sponge-like diffuse retinal thickness (SLDRT) rather than subretinal fluid (SRF) on OCT images could help the clinician identify DME patients with potential best corrected visual acuity (BCVA) decline (decimal notation 0.5 cut-off). Through CNN feature extraction, the machine can identify collective morphology predictors and perform precise disease dimensionality reduction. Consequently, AI proved its utility in disease classification advancement, severity staging, dual-core treatment decisions, and prognosis prediction. Still, it is challenging to achieve disease early diagnosis, especially the subclinical microvascular changes in early retinopathy and their clinical capability in predicting leakage points. To fully explore the diagnostic power of OCTA images, we extended our previous OCT-AI building experience and established a novel NV detection method. This invention was also conducted with a relatively modest sample size for each group. Several significantly defective images with segmentation errors and images from patients with macular edema were excluded to minimize the effect of errors in our invention. Other exclusion criteria included eyes with prior history of vitreoretinal surgery, intravitreal injections, or macular hemorrhages greater than a typical blot hemorrhage. A much larger and multi-centered OCTA database can hence be used to validate our future studies system further to consider its future clinical implementation. The system's diagnostic accuracy can also be further enhanced by incorporating the patients' medical history and other clinical information in the screening tool. Nonetheless, we have developed an AI-based screening tool with minimal processing time. In contrast to the weeks of scheduling time required between OCT and FAG imaging, an AI-based OCTA evaluation during the first visit to the ophthalmologist alone is sufficient to determine the patient's prognosis and treatment plan. It requires only 4-6 seconds to extract angio-biomarkers from each OCTA image.

Considering the prospects of cloud-based systems used by ophthaImologists31, the AI-OCTA system according to the invention can potentially be implemented in the form of a web-based interface. It was motivated by the integration of concepts of cloud computing and telemedicine with AI in diagnosing AMD. It has been demonstrated that smart healthcare practices may lead to improved accuracy of diagnostic tools and henceforth more effective patient care. The system according to the invention can analyze OCTA images to classify AMD types and provide medical recommendations. In other words, anyone with a computer and the Internet connection can make use of our AI model. The AI-OCTA system we have developed is not only a prompt detection module, but also an effective alternative to FAG/ICG in characterizing neovascularization (NV) activity. It may reduce the workload of healthcare professionals, and patients can have access to their diagnostic reports immediately to decide if they should seek further treatment. This is also an advantageous next-generation diagnostic solution that can be useful in remote places with less medical services. In its potential future applications in clinical settings, less human power will be needed to run an AMD diagnostic protocol to achieve an accuracy nearly as ideal as the referential FAG/ICG examination. Overall, it is hoped that continued development and refinement to the AI-OCTA system will result in its eventual application in clinical settings. Besides overcoming the limitations faced by the individual imaging techniques of FAG, ICG, OCT, and OCTA, the system can also improve an AMD patient's overall diagnosis and treatment experience.

It can be concluded that the diagnostic method of angiography is time-consuming and may require multiple injections justified the need for a novel, non-invasive dye-independent approach to angiography. Such novel method, OCTA, allows localization and description of vascular lesions using both structural and blood flow information, resolving vascular trees layer by layer and depicting NV sprouting at a 5 μm resolution. Taking it a step further, AI has proven its potential in utilizing OCTA images to improve the efficiency and accuracy of diagnosing AMD vasculopathy at early stages. By applying AI for OCTA analysis, its future application in clinical setting means that ophthalmologists can diagnose AMD using a protocol with reduced workload and time without compromising high accuracy. Overall, this is a pivotal invention that lays the foundation for future applications of AI to work in novel optic images and diagnostics modules.

The following embodiments are made to clearly exhibit the above-mentioned and other technical contents, features, and effects of the present invention. As the contents disclosed herein should be readily understood and can be implemented by a person skilled in the art, all equivalent changes or modifications which do not depart from the concept of the present invention should be encompassed by the appended claims.

### Examples

I. Materials and Methods

Ethical and Information Use Approval

The collection of retrospective data and their manipulations were performed under the Institute Review Board of Taipei Veterans General Hospital's approval. De-identification was performed according to the Big Data Center, Taipei Veterans General Hospital (BDC, TVGH) protocol. All retrospective clinical information and data were de-identified before undertaking research.

Demographics, Classification and Annotation of the Study Population

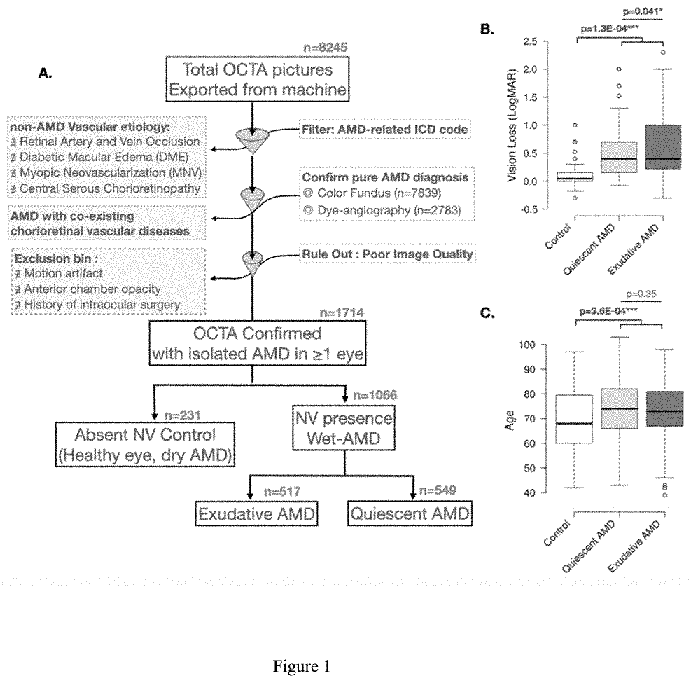

OCTA imaging and other associated medical records used in this invention were primarily collected from patients who had been diagnosed with exAMD and received treatment at the Department of Ophthalmology, Taipei Veterans General Hospital between January 2017, and December 2020. Baseline demographic characteristics of our cohort includes age, gender, best corrected vision acuity (BCVA), OCT-angiography images (Optovue RTVue-XR Avanti), OCT scans (Optovue RTVue-XR Avanti), fluorescent angiography (FA) and history of intra-vitreous anti-VEGF injection (FIG. 1, (A)). A total 8245 OCTA pictures were retrieved from the OCTA machine by matching the electronic medical records database using AMD-related diagnosis codes of International Classification of Diseases, Tenth Revision (ICD-10). To present to the model with pure AMD cases, non-AMD etiology that may cause macular lesions such as retinal vessels occlusion, diabetic macular edema, myopic neovascularization, or central serous chorioretinopathy on OCTA scans were filtered out by negative selecting corresponding ICD-10 codes. Since dye-based angiography remains the gold standard for diagnosis of exAMD, an additional 7839 color fundus pictures and 2783 FA and indocyanine green angiography were applied to further examined for AMD lesions, such that the OCTA pictures with other chorioretinal vascular diseases would not confound model establishment. Next, poor-quality images due to media opacity, severe motion, shadowing, or significant artifacts on OCTA imaging were removed. After the initial exclusions, a total net count of 1714 OCTA pictures were finally enrolled for this study. Two retinal fellows were recruited to perform the annotation task by confirming AMD diagnosis. The fellows labeled the cases by whether a neovascular plexus was present, and whether the neovascular plexus was in active status with exudation. AMD cases with the presence of NV was defined as wet AMD (n=1066). Wet AMD is further classified as active AMD (n=517) if the NV was denoted by a leakage on FA/ICGA or retinal fluid on OCT, otherwise as quiescent AMD (qAMD n=549). Images with non-detectable NV was further divided into normal or dry AMD (drusens). The fellows were informed with all the necessary information necessary to discriminate AMD types, including OCT, color fundus photography, FAG images, and clinical records. The annotated images then went through the second round of review by an expert ophthalmologist to ensure annotation quality. If questionable, the annotated image was peer-reviewed by three ophthalmologists to conclude whether it should remain in the dataset. All discernible information, such as the patient's name, birth date, ID number, were removed, and images were assigned a random serial number.

Best-corrected visual acuity (BCVA) was compared among the four groups, wetAMD cases had significantly poorer BCVA than control (logMAR: 0.54±0.49 vs 0.1±0.2, p=1.3E-04***) and patients with exudative AMD had worse BCVA than the quiescent AMD (logMAR: 0.57±0.48 vs 0.51±0.49, p=0.04*) (FIG. 1, (B)). Meanwhile, the age distribution between exAMD and qAMD was insignificant (73.4±11.8 vs 72.7±10.9, p=0.35) (FIG. 1, (B)).

Development of AMD classification network by deep learning

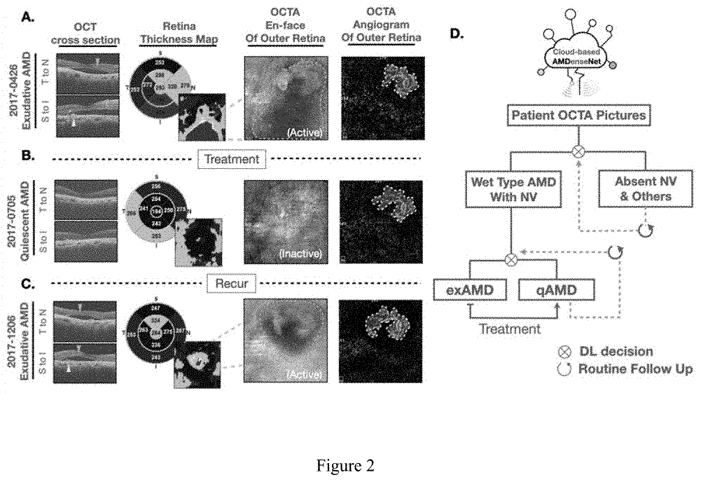

The presentation of exAMD features can be depicted by variable image modalities. However, OCT sometimes captures false negative fluid scans and dye leakage in FA obscure microvascular structures and produce dimension reduction problems. (FIG. 2, (A)). Through serial IVI anti-VEGF treatment, these features may respond as regression or maintenance. (FIG. 2, (B)). Whenever disease recurs, subtle features change could present before clinical observation (FIG. 2, (C)). The three-dimensional imaging in OCTA is especially useful for characterizing layer-specific vascular lesions that ideally suited for monitoring NV progression and treatment response. We thereby schemed a diagnostic loop-work that could be able to reflect the disease status. (FIG. 2, (D)).

The Image Acquisition and Processing of OCTA

OCTA volumes of 3 mm×3 mm macular area were obtained via the split-spectrum amplitude decorrelation angiography algorithm with a resolution consisting of 304×304 A-scans. The raw OCTA images were acquired from the OptoVue device, image size 3499*2329 pixels, resolution 96 dpi, and the bit depth was set as 24. The OCTA acquisition generated en-face and angiogram images, which were auto-segmented to depict the superficial capillary plexus (SCP), deep capillary plexus (DCP), outer retinal layer, and the choroid capillary layer from the OCTA built-in software. After the initial collection, the region of interest (ROI) preprocessing was executed by auto-alignment and cropping the raw OCTA images using our customized pre-process algorithm and the resultant ROI was 757*757 pixels before loaded to the model channel.

The Retinal Images of the Early and Late Stages of AMD

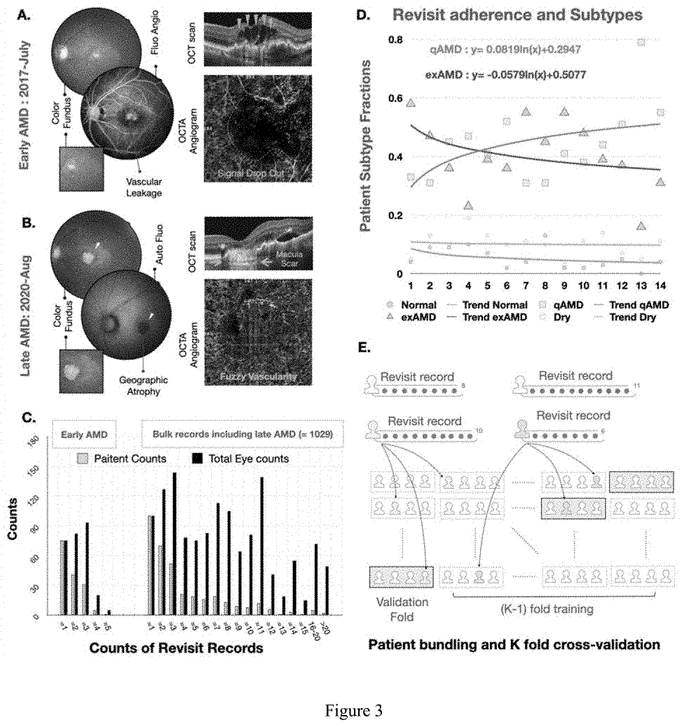

Neovascular leakages and consequent damages may cause retinal remodeling. For instance, the image set in (FIG. 3, (A)) depicts the retina features in early AMD. However, the status post three years of exAMD recurrence, geographic epithelium atrophy was denoted by fundus pictures and fibrotic scars were also prominent in both OCT and OCTA images, thereby blurring the essential vascular and structural information a retina photo provided within (FIG. 3, (B)) and could interfere with image classification by deep learning.

In respond to AMD evolution, early and late AMD subgroups were setup to test model generality, which we defined the early AMD as pictures taken one year within the first diagnosis and the late AMD as examination beyond one year (FIG. 3, (C)). Judging from our in-house revisit record, the degree of revisit adherence reverse correlates with the fraction of exAMD patients (FIG. 3, (D)). To facilitate DL, acquire consecutive NV change in a similar retina background, and to prevent cross contamination in train-validation dataset we performed patient bundling in the context of K-fold experiments (FIG. 3, (E)). The dataset images were randomly distributed into a training set (80%) and a validation set (20%). To overcome the limitation of the dataset size and enhance our AI model's performance, we employed augmentation to the training dataset, including one random horizontal flip and two random crops of the images, thus enhancing their variation.

Model Development and Training of AI Model

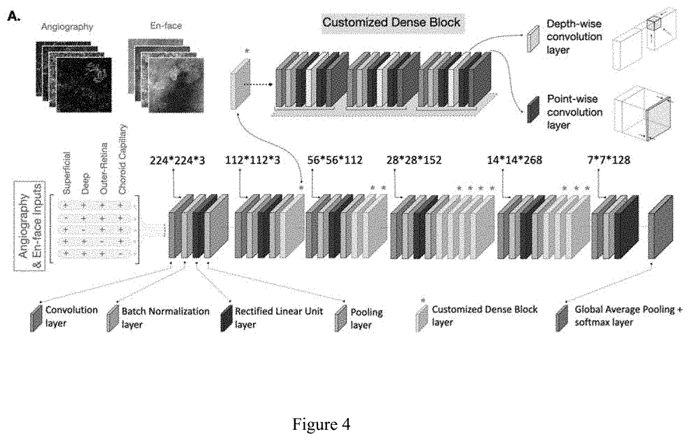

Referring to FIG. 4, in this study we used DenseNet-121 convolutional neural network (CNN) to classify OCTA images. Given that we used different input combinations, either single image or combinations of up to 4 images, we modified the basic DenseNet-121 architecture to suit these different input types. Whereas the conventional input in the CNN consists of 3 channels for RGB colors, for image combinations, the number of input channels was modified to the multiples of 3. Due to the relative scarcity of the data, the 10-fold cross-validation was applied to observe the trained AI model's objective performance. The original images were bilinearly down sampled to resolution 448×448, and pixel values were normalized from 0-255 to the range of 0-1. Intensive data augmentation was used during training, including random horizontal flip, random scaling from 90% to 110%, and random crop. The numbers such as “224*224*3” recited in FIG. 4 refers to size of compressed image multiplies by number of channels. The compressed image is obtained through convolutional feature extraction of the original image. For example, “224*224*3” means a compressed image size of 224*224 with three channels of colors (e.g., R, G, B) or features (e.g., contrast, granularity, connectivity). The adaptive moment estimation (Adam) and categorical cross-entropy were applied as the optimizer and the loss function. In the equation below: mₜ and vₜ indicates the 1^{st} and 2^{nd} vector; β₁ and β₂ indicates the exponential decay rate for the moment estimates; and gₜ and gₜ² indicates the element-wise and the element-wise square with the time step. The default settings were β1=0.9, β2=0.999. The total training epoch was set as 600 iterations based on the learning rate with 1e-3, and batch size has been set as 8 per step. The AI models were established using the Ubuntu 16.04 LTS operation system with the GeForce RTX 2080 Ti graphic processing unit (GPU) card, whereas Keras 2.2.4 and TensorFlow-GPU 1.6.0 software were used for training and validation.

mₜ=β₁mₜ₋₁+(1

vₜ=β₂vₜ₋₁+(1

Verification of AI Models and Data Analyses

To verify our AI model, the confusion matrix was applied to compare between ground truth (ophthalmologist's annotation) and the AI's performance. Based on the ground truth empirical (ophthalmologist's annotation) and the AI model prediction result, the confusion matrices were applied to present clinical verification results. The confusion matrix includes two major parameters, AI prediction result, and ground truth. Each major parameter contained two minor parameters, the predicted result (positive, P and negative, N) and ground truth (true, T and false, F). Those minor parameters integrated a 2*2 matrix, which includes 4 categories, true positive (TP), false positive (FP), false negative (FN), and true negative (TN). According to the confusion matrix, we can also calculate the recall, precision, accuracy, and F1-score, which were common and standard parameters for evaluating biomedical image recognition performance.

accuracy=(TP+TN)/(TP+FP+TN+FN)

precision=TP/(TP+FP)

recall=TP/(TP+FN)

F1-socre=2*(precision*recall)/(precision+recall)

Statistics

Analysis of variance (ANOVA) has been applied to compare the difference of dataset demographics between groups. The dataset demographics include age and best corrected visual acuity (BCVA).

The model's performance in detecting AMD classification and NV activity was evaluated by area under the receiver operating characteristic curve (AUROC) and confidence intervals. Kappa score was used for measurement of inter-layer agreement. The level of significance was set at α<0.05. Statistical analyses were performed using the SPSS 20.0.

II. Results

Deep-Learning Model Performance and Classification Decisions

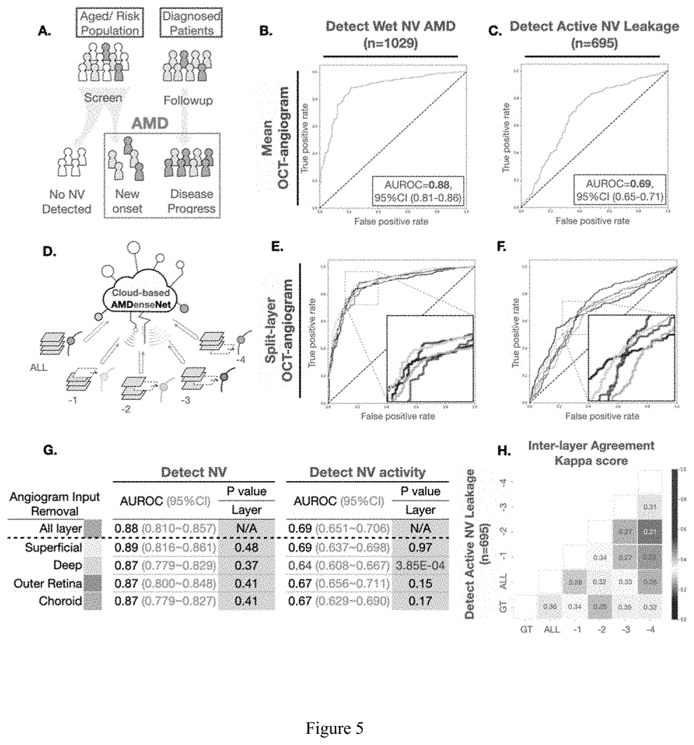

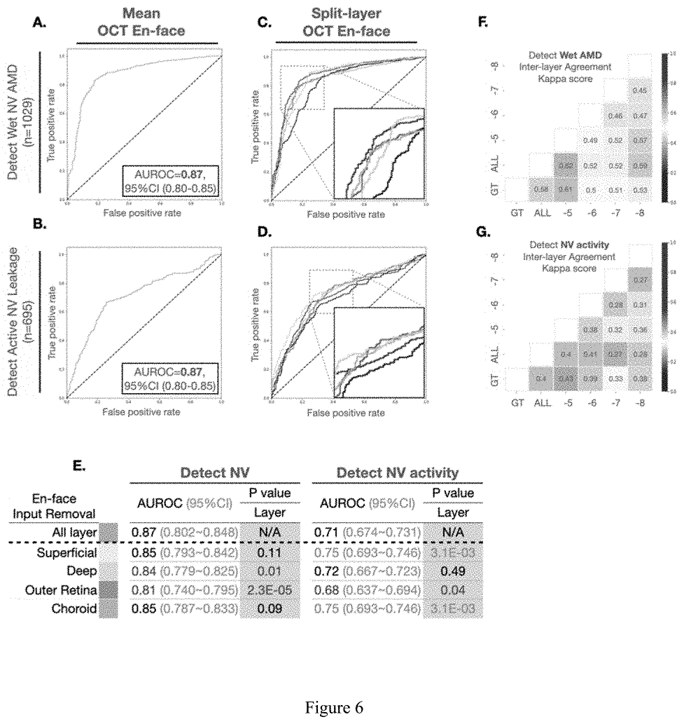

To screen and characterize referable AMD from the general population, we developed deep learning models interpretable of abnormal vascular lesions from retinal angiography (FIG. 5, (A)). Our model showed a good discriminatory ability in detecting the presence of NV and NV activity from a combination of the four angiogram inputs (AUROC: 0.88 and 0.69 respectively) (FIG. 5, (B), (C)). To further investigate model dependency on the anatomy plexus, we conducted inter-rater agreement tests and neovascular classification with an array of input combinations, whereby the ΔDL kappa score and ADL performance (full layer input—specific layer removed input) may indicate the anatomy significance underlying model decisions (FIG. 5, (D)). When we dissected the general curve into sub-experiments representing split layer removal, we derived partitioned ROC course shown as in (FIG. 5, (E), (F)). By calculating the significance of each split layer from the AUROC, we concluded that the DCP angiogram removal causes significant AUROC drop in NV activity detection (p=3.8E-04***) (FIG. 5, (G)). In align to this result, we observed model consistency reduction was more associated to input removal of the DCP (FIG. 5, (H)). In parallel, experiments were repeated with the En-face modality inputs from the same dataset (FIG. 6, (A)-(D)), in which the En-face modality generated angiogram comparable performance in detecting NV presence and its activity (AUROC: 0.87 and 0.71 respectively). However, the model relies more upon the outer retina structural anatomy to make equitable (FIG. 6, (E)) and consistence (FIGS. 6, (F) & (G)) decision on NV exudation status. While the angiographic DCP and en-face outer retina both affects DL performance, this provided an argument that the thinking process of DL was affected by anatomy information but in a context dependent phenomenon.

Deep Learning Stratifies AMD Risk with Specific Vascular Layer Features and in Association with Visual Loss

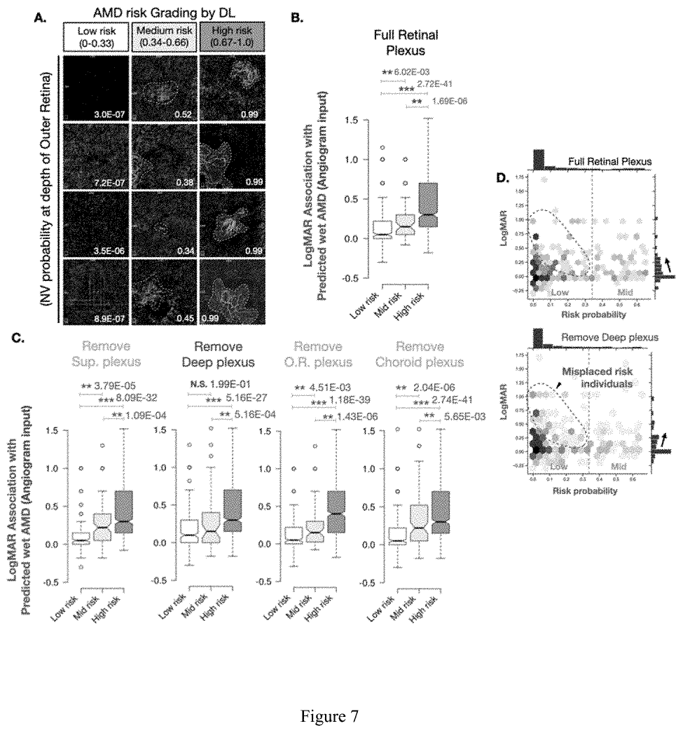

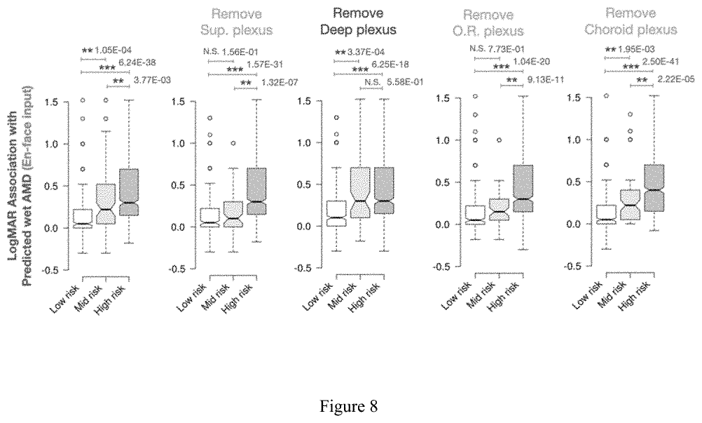

The derived model grades the tested subject by a 3-tier risk, in which the higher the graded risk matches to an increase NV anatomical size and maturity (FIG. 7, (A)). Correspondingly, the scale of AMD risk was aligned to the degree of patient vision loss. (LogMARLow risk vs Mid risk, p=6.02E-03**; LogMARMid risk vs High risk, p=1.69E-06***) (FIG. 7, (B)). However, when we remove the DCP angiography inputs, the significant difference of visual acuity between the low and mid risk groups were diminished (p=1.99E-01) (FIG. 7, (C)). By comparing the hex-bin plot distribution, we denoted a shifted result in the DCP angiogram removal experiment, wherein a subpopulation of poor vision was misplaced to the low-risk AMD group (FIG. 7, (D)). In align to this finding, the DCP in en-face inputs were also indispensable to predict a significant visual loss between the Mid and High-risk AMD (LogMARMid risk vs High risk: en-face full input, p=3.77E-03**; deep removal, p=5.58E-01) (FIG. 8). To investigate misclassification of vision loss in DCP removal, we calculated the risk in each layer combinations. While the low risk logMAR all angiogram layer was 0.13±0.26, the low risk logMAR DCP removal was significant higher 0.18±0.29 (p=0.05*). Similarly, the mean logMAR prediction in the low-risk AMD was also higher when the DCP was removed from the en-face inputs (logMAR all en-face layer=0.15±0.28, logMAR DCP removal=0.22±0.35, p=0.005**). Together, the removal of either the angiogram or en-face inputs of DCP made the model prone to over-estimate the patient vision loss, while such crippling effect was not observed in other layer removal. This indicated that the distinct anatomy withheld asymmetric information which influence differentially to the model decision and classification.

Testing Model Generality in the Early and Late Phases of AMD

The image characteristics of AMD evolved among status pre- and post-anti-VEGF treatment; thus, the vascular features could be heterogenous throughout a span of clinical course (FIGS. 3, (A) & (B)). Hence, we created a scoring matrix to evaluate the model generality in the context of early and late AMD. Particularly, when detecting NV presence, the model preserves a comparable level of sensitivity (91.9% vs. 88.48%, p=0.437) between AMD stages, but dropped significantly in the measure of accuracy (p=0.016*), specificity (p=0.016*), precision (p=0.006**) and F1-score (p=0.017*) in the late AMDs. Similarly, the model sensitivity in exAMD detection was also not affected by stages of AMD (77.53% vs 79.64%, p=0.837), whereas the accuracy and specificity dropped significantly. Together, the approximate sensitivity score measured across disease stages indicated that the model was able to attribute true positive cases from variable anatomy presentations.

Comparing Model Applicability with Real-World Inspection Standards

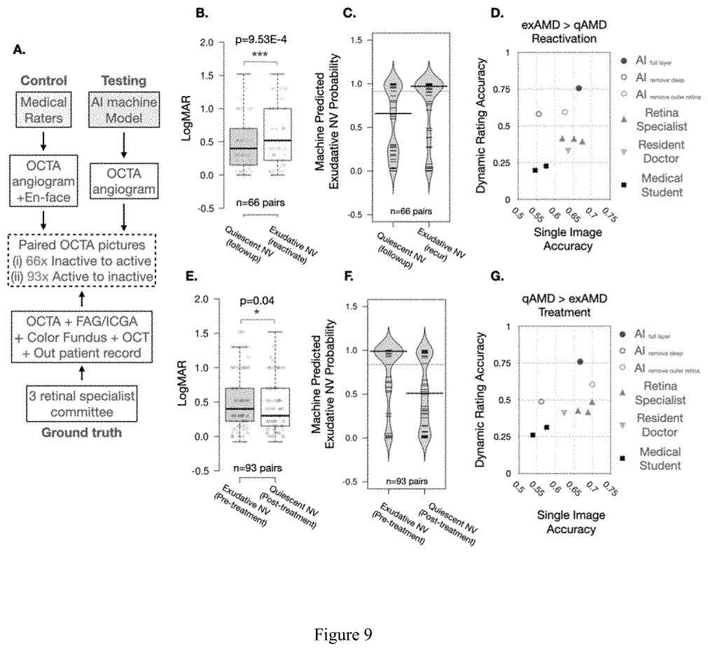

In order to examine the applicability of AI in clinic scenarios, we enroll retinal specialists and other medical associate individuals to match AI in the specified paired NV transition (exAMD-to-qAMD and qAMD-to-exAMD) tests (FIG. 9, (A)). For the 66 pair reactivation cases, vision acuity decreased significantly (p-value=9.53E-4***) after NV reactivation (FIG. 9, (B)), meanwhile the median of AI predicted probability also raises from 0.67 to 0.96 (FIG. 9, (C)). Interestingly, while characterizing the sequential change of the paired pictures, AI (accuracy=0.663) scored similarly to three retina specialists (mean=0.648; SD=0.027) in single image test but is more sensitive (accuracy=0.760) than the retinal specialists (mean=0.403; SD=0.011) in capturing the dynamic changes of NV reactivation (FIG. 9, (D)). In align to our previous result, the removal of DCP and outer retina layer significantly affects the model predictions. On the other hand, in the 93 pair of treatment remission cases, vision loss logMAR reduces significantly (p-value=0.04*) after anti-VEGF treatment (FIG. 9, (E)), and the AI predicted probability median dropped from 0.98 to 0.51 (FIG. 9, (F)). The AI scores (accuracy=0.666) near to the retina specialists (mean=0.683; SD=0.020) in single image test, AI is more sensitive (accuracy=0.761) than the retinal specialists (mean=0.463; SD=0.038) in capturing the dynamic changes of NV remission (FIG. 9, (G)). Wherein we noted that the removal of DCP but not the outer retina could affect model prediction. Worth to mention, we included medical students in the process of paired transition test. Despite the medical students were adequately educated with the OCTA features in exAMD and quiescent AMD, they tradition accuracy rate was around 0.25, which is arguably the expected value of 0.5×0.5 in the null guess.

Implement DL Model to the Longitudinal Follow-Up of Recurrent NV Activity

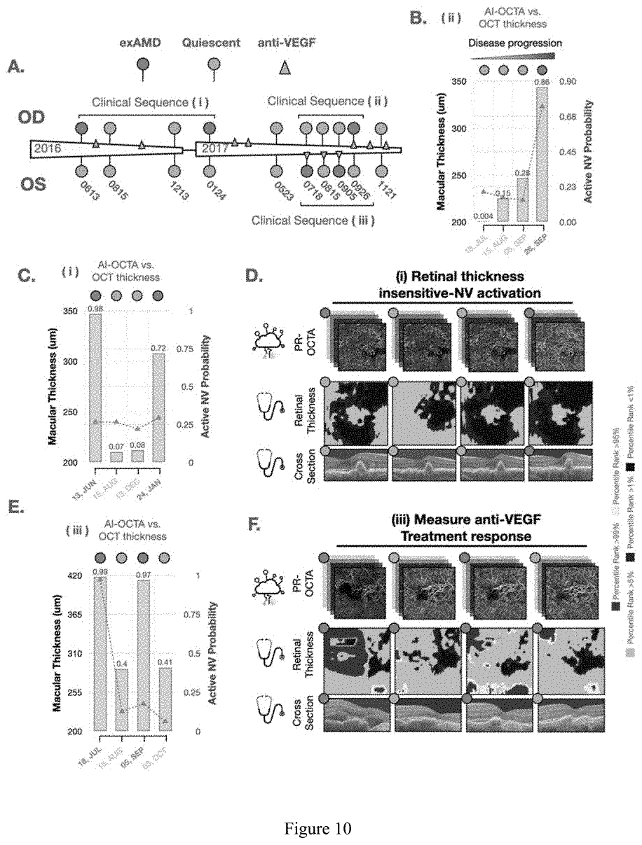

The on and off nature of NV relapse leakage was clinically challenging to be followed. Here we detailed a patient with longitudinal revisit and treatments to test our model performance on the consecutive transitions in three clinical sequences we have specified (FIG. 10, (A)). Beside the goal to validate the predictions, we also aimed to investigate the discrepant sensitivity of diverse modalities commonly used to assess disease activity. For instance, in this subclinical NV case, the DL depicts a trend of rising exAMD probability while the retinal thickness remains unchanged (FIG. 10, (B)). In support to this finding, the four revisit records of clinical sequence (i) depict a treat and recur scenario, wherein the NV leakage did not cause significant thickness changes of the retina but was still correctly identified by the DL-OCTA (FIG. 10, (C)). Moreover, by judging the right eye (OD) exAMD event on 13 Jun. 2016, it can be mis-classified by both the cross OCT and retinal thickness map results (FIG. 10, (D)). In the other context such as clinical sequence (iii), the retinal thickness changes in accord to the exuding status and DL-OCTA prediction results (FIG. 10, (E)), but the absence of fluid could cause misinterpretation to the clinical raters (FIG. 10, (F)). From this point, we have demonstrated the robustness of DL characterizing the relapse feature of NV.

Axiom Attribution Guides Interpretable Attention to Neovascular Feeder Vessels

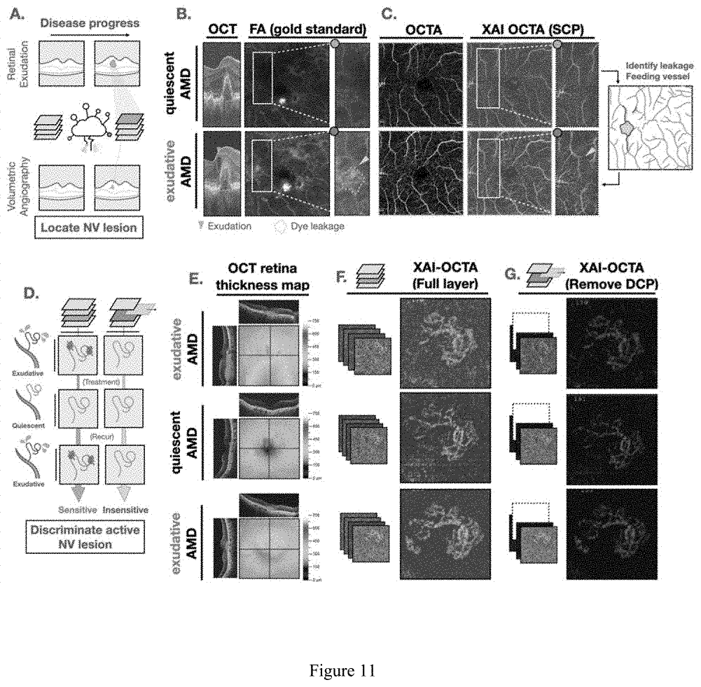

Lastly, we applied axiom attribution (method) to distinguish the vascular regions that DL could have used to make neovascular assessments. Two representative cases were enrolled to support DL being competent to attribute interpretable attention to layer specific and branch specific vascular leak points (FIG. 11, (A)). In a conversion case of exAMD, a retinal exudation was noted at the superficial layer. The exudation was further confirmed by the dye leakage (circled by the blue dashed line) on fluorescent angiography (FA) (FIG. 11, (B)). Interestingly, when being analyzed by DL-OCTA, we command machine to attribute attention to the superficial capillary plexus (SCP), thereby we observed a heatmap with color-coded (red/blue) indicate pixels to be increased/reduced in intensity to attain higher attention overlaying to the peri-exudation vessel skeleton (illustrated in the cartoon on the right) (FIG. 11, (C)). To exclude the possibility of random attribution from the feeder vessel identification, we further incorporated the DCP removal experiment, to validate both the DCP vascular significance as well as the explain ability of our model (FIG. 11, (D)). First, we confirmed the exAMD-qAMD-exAMD sequence by clinical checkups (FIG. 11, (E)) and locate the subretinal fluid. We then rerun the attention attribution model with input full layer on the outer retina of OCTA, which we found attention was attributed to the vascular meshwork when the NV was exudative and was otherwise un-annotated when the NV was quiescent (FIG. 11, (F)). Encouragingly, when we performed same attention model to the result of input DCP removal, we surprisingly found the model went blind to the deja-vu vascular features and could not further discriminate the exudative or quiescent state of the AMD (FIG. 11, (G)). Together, the results of our model suggested a tight dependency between pathology recognition and disease classification.

It was confirmed in this study that angiogram information alone can identify wet-NV activity as effective as the structural en-face data (F1 score=91.7). In two long term follow-up cases, XAI marks off bare eye invisible microvascular lesions before visual decline and on-site NV formation, early detection was made with a heralding time of 3 and 6 months.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

1. Wong, T Y, et al., The natural history and prognosis of neovascular age-related macular degeneration: a systematic review of the literature and meta-analysis. Ophthalmology, 2008. 115(1): p. 116-26.
2. Kodjikian, L., et al., Fluid as a critical biomarker in neovascular age-related macular degeneration management: literature review and consensus recommendations. Eye (Lond), 2021. 35(8): p. 2119-2135.
3. Willoughby, A. S., et al., Subretinal Hyperreflective Material in the Comparison of Age-Related Macular Degeneration Treatments Trials. Ophthalmology, 2015. 122(9): p. 1846-53 e5.
4. Inoue, M, et al., A Comparison Between Optical Coherence Tomography Angiography and Fluorescein Angiography for the Imaging of Type 1 Neovascularization. Invest Ophthalmol Vis Sci, 2016. 57(9): p. OCT314-23.
5. Siggel, R., et al., Optical coherence tomography angiography for the detection of macular neovascularization-comparison of en face versus cross-sectional view. Eye (Lond), 2022.
6. De Fauw, J., et al., Clinically applicable deep learning for diagnosis and referral in retinal disease. Nat Med, 2018. 24(9): p. 1342-1350.
7. Hwang, D. K, et al., Artificial intelligence-based decision-making for age-related macular degeneration. Theranostics, 2019. 9(1): p. 232-245.
8. Lee, C. S., D. M. Baughman, and A. Y. Lee, Deep learning is effective for the classification of OCT images of normal versus Age-related Macular Degeneration. Ophthalmol Retina, 2017. 1(4): p. 322-327.
9. Son, J., et al., Development and Validation of Deep Learning Models for Screening Multiple Abnormal Findings in Retinal Fundus Images. Ophthalmology, 2020. 127(1): p. 85-94.
10. Tham, Y C., et al., Referral for disease-related visual impairment using retinal photograph-based deep learning: a proof-of-concept, model development study. Lancet Digit Health, 2021. 3(1): p. e29-e40.
11. Yim, J., et al., Predicting conversion to wet age-related macular degeneration using deep learning. Nat Med, 2020. 26(6): p. 892-899.
12. Hormel, T T, et al., Artificial intelligence in OCT angiography. Prog Retin Eye Res, 2021. 85: p. 100965.
13. Marques, J. P., et al., Sequential Morphological Changes in the CNV Net after Intravitreal Anti-VEGF Evaluated with OCT Angiography. Ophthalmic Res, 2016. 55(3): p. 145-51.
14. Mathis, T, et al., Retinal Vascularization Analysis on Optical Coherence Tomography Angiography before and after Intraretinal or Subretinal Fluid Resorption in Exudative Age-Related Macular Degeneration: A Pilot Study. J Clin Med, 2021. 10(7).
15. Hagag, A. M., et al., OCT Angiography Changes in the 3 Parafoveal Retinal Plexuses in Response to Hyperoxia. Ophthalmol Retina, 2018. 2(4): p. 329-336.
16. Nesper, P. L., et al., Hemodynamic Response of the Three Macular Capillary Plexuses in Dark Adaptation and Flicker Stimulation Using Optical Coherence Tomography Angiography. Invest Ophthalmol Vis Sci, 2019. 60(2): p. 694-703.
17. Au, A., et al., Volumetric Analysis of Vascularized Serous Pigment Epithelial Detachment Progression in Neovascular Age-Related Macular Degeneration Using Optical Coherence Tomography Angiography. Invest Ophthalmol Vis Sci, 2019. 60(10): p. 3310-3319.
18. Bonini Filho, M A., et al., Association of Choroidal Neovascularization and Central Serous Chorioretinopathy With Optical Coherence Tomography Angiography. JAMA Ophthalmol, 2015. 133(8): p. 899-906.
19. Tan, B., et al., Quantitative Microvascular Analysis With Wide-Field Optical Coherence Tomography Angiography in Eyes With Diabetic Retinopathy. JAMA Netw Open, 2020. 3(1): p. e1919469.
20. Arrigo, A., et al., Macular neovascularization in AMD, CSC and best vitelliform macular dystrophy: quantitative OCTA detects distinct clinical entities. Eye (Lond), 2021. 35(12): p. 3266-3276.
21. Ma, K., et al., ROSE: A Retinal OCT-Angiography Vessel Segmentation Dataset and New Model. IEEE Trans Med Imaging, 2021. 40(3): p. 928-939.
22. Elnahry, A. G. and D. J. Ramsey, Automated Image Alignment for Comparing Microvascular Changes Detected by Fluorescein Angiography and Optical Coherence Tomography Angiography in Diabetic Retinopathy. Semin Ophthalmol, 2021. 36(8): p. 757-764.
23. Martinez-Rio, J., et al., Robust multimodal registration of fluorescein angiography and optical coherence tomography angiography images using evolutionary algorithms. Comput Biol Med, 2021. 134: p. 104529.
24. Tan, A. C. S., et al., An overview of the clinical applications of optical coherence tomography angiography. Eye (Lond), 2018. 32(2): p. 262-286.

What is claimed is:

1. A computer-implemented method for diagnosing age-related macular degeneration (AMD), the method comprising:
receiving one or more optical coherence tomography angiography (OCTA) image of a subject;
pre-processing the one or more OCTA image to obtain image data;
inputting the image data to a trained deep learning (DL) network;
generating, using the trained DL network, an output that characterizes the health of the subject with respect to AMD; and
generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

2. The method of claim 1, wherein the one or more OCTA image is an en-face OCT image or an OCTA angiogram.

3. The method of claim 2, wherein the pre-processing comprises segmenting the OCTA image to obtain at least one of an image of superficial capillary plexus, an image of deep capillary plexus, an image of outer retinal layer, and an image of choroid capillary layer.

4. The method of claim 3, wherein the output is generated based on image data of at least the image of deep capillary plexus.

5. The method of claim 4, wherein the output is generated based on image data of at least the image of deep capillary plexus and the image of outer retinal layer.

6. The method of claim 1, wherein a plurality of training OCTA images is used in training the DL network, each training OCTA images being pre-processed by segmenting training OCTA image to obtain at least one of an image of superficial capillary plexus, an image of deep capillary plexus, an image of outer retinal layer, and an image of choroid capillary layer.

7. The method of claim 6, wherein the DL network is trained with image data of the image of superficial capillary plexus, the image of deep capillary plexus, the image of outer retinal layer, and the image of choroid capillary layer.

8. The method of claim 1, wherein the classification of AMD classifies the subject as having no AMD, wet AMD or dry AMD.

9. A system comprising one or more computers and one or more storage devices storing instructions that when executed by the one or more computers cause the one or more computers to perform operations comprising:
receiving one or more optical coherence tomography angiography (OCTA) image of a subject;
pre-processing the one or more OCTA image to obtain image data;
inputting the image data to a trained deep learning (DL) network;
generating, using the trained DL network, an output that characterizes the health of the subject with respect to AMD; and
generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

10. The system of claim 9, wherein the one or more OCTA image is an en-face OCT image or an OCTA angiogram.

11. The system of claim 10, wherein the pre-processing comprises segmenting the OCTA image to obtain at least one of an image of superficial capillary plexus, an image of deep capillary plexus, an image of outer retinal layer, and an image of choroid capillary layer.

12. The system of claim 11, wherein the output is generated based on image data of at least the image of deep capillary plexus.

13. The system of claim 12, wherein the output is generated based on image data of at least the image of deep capillary plexus and the image of outer retinal layer.

14. The system of claim 9, wherein a plurality of training OCTA images is used in training the DL network, each training OCTA images being pre-processed by segmenting training OCTA image to obtain at least one of an image of superficial capillary plexus, an image of deep capillary plexus, an image of outer retinal layer, and an image of choroid capillary layer.

15. The system of claim 14, wherein the DL network is trained with image data of the image of superficial capillary plexus, the image of deep capillary plexus, the image of outer retinal layer, and the image of choroid capillary layer.

16. The system of claim 9, wherein the classification of AMD classifies the subject as having no AMD, wet AMD or dry AMD.

17. One or more non-transitory computer-readable storage media encoded with instructions that when executed by one or more computers cause the one or more computers to perform operations comprising:
receiving one or more optical coherence tomography angiography (OCTA) image of a subject;
pre-processing the one or more OCTA image to obtain image data;
inputting the image data to a trained deep learning (DL) network;
generating, using the trained DL network, an output that characterizes the health of the subject with respect to AMD; and
generating, based on the output, a diagnostic result comprising an indication of presence of neovascularization (NV) or presence of NV activity in the subject, an identification of a location of NV or NV activity or a feeder vessel supplying for an NV exudation in the one or more OCTA image, a numerical value representing a probability that the subject has AMD, a classification of AMD in the subject, or a combination thereof.

18. The computer-readable storage media of claim 17, wherein a plurality of training OCTA images is used in training the DL network, each training OCTA images being pre-processed by segmenting training OCTA image to obtain at least one of an image of superficial capillary plexus, an image of deep capillary plexus, an image of outer retinal layer, and an image of choroid capillary layer.

19. The computer-readable storage media of claim 18, wherein the DL network is trained with image data of the image of superficial capillary plexus, the image of deep capillary plexus, the image of outer retinal layer, and the image of choroid capillary layer.

20. The computer-readable storage media of claim 17, wherein the classification of AMD classifies the subject as having no AMD, wet AMD or dry AMD.
